(19) United States
(12) Patent Application Publication
(10) Pub. No.: US 2003/0232767 A1
(43) Pub. Date: Dec. 18, 2003

(54) Sensitization of cells to cytotoxic agents using oligonucleotides directed to nucleotide excision repair or transcritpion coupled repair genes

(76) Inventors: Sudhir Agrawal, Shrewsbury, MA (US); Ekambar R. Kandimalla, Southboro, MA (US); David B. Bregman, Trumbull, CT (US); Sridhar Mani, Riverdale, NY (US); Yi Lu, Bronx, NY (US)

Correspondence Address: HALE AND DORR, LLP, 60 STATE STREET, BOSTON, MA, 02109

(21) Appl. No.: 09/825,489

(22) Filed: Apr. 3, 2001

(51) Int. Cl. A61K 48/00; A61K 31/28; A61K 33/24; A61K 33/40

(52) U.S. Cl. 514/44; 424/649; 424/616; 514/492; 600/1

(57) ABSTRACT

This invention relates to the fields of molecular biology and oncology. More particularly, this invention relates to the sensitization of cancerous cells to therapeutic agents. The invention provides methods, compositions, and formulations for potentiating or enhancing the toxicity of various cytotoxins and oxidizing agents, and of reducing the resistance and proliferation rate of cancer cells. It also provides various compositions and therapeutic formulations useful as anticancer agents.

### FEDERALLY SPONSORED RESEARCH

This work was supported by Grant 96-59 from the James S. McDonnell Foundation New Investigator Program (DBB), RO1 CA80171-01 from the NCI (DBB), a pilot award from the American Cancer Society (SM), and Cancer Center Core grant 5-P30-CA13330-26 from the NIH.

### BACKGROUND OF THE INVENTION

1. Field of the Invention

This invention relates to the fields of molecular biology and oncology. More particularly, this invention relates to the sensitization of cancerous cells to therapeutic agents.

2. Summary of the Related Art

Nucleotide excision repair (NER) is essential for the removal of a variety of helix distorting DNA lesions, including those induced by UV radiation and the anticancer agent cisplatin (de Laat et al. (1999) Genes Dev. 13:768-85; Reed (1998) in Cancer Treat Rev., Vol. 24, pp. 331-44). Individuals with the sun sensitivity/skin cancer predisposition syndrome, Xeroderma pigmentosum (XP), may have defects in one of seven key NER proteins (XPA-XPG). At least 20 additional gene products are required for NER (de Laat et al. (1999) Genes Dev. 13:768-85; Wood (1997) J. Biol. Chem. 272:23465-8). Transcription coupled repair (TCR) refers to the expedited repair of lesions located on the transcribed strand of active genes either by NER or by base excision repair, which removes oxidative lesions. In TCR, lesion recognition is assisted by the stalling of RNA polymerase II (RNAP II) at the lesion (reviewed in de Laat et al. (1999) Genes Dev. 13:768-85). Individuals with Cockayne syndrome (CS) have a mutation in either of two proteins, Cockayne syndrome group A (CSA) or Cockayne syndrome group B (CSB). Such mutations lead to deficient TCR and the clinical features of CS which include short stature, cachexia, and sun sensitivity, but surprisingly no predisposition to developing cancer.

It has been proposed that the products of the CSA and/or CSB gene recruit the NER apparatus to sites of stalled RNAP II to permit rapid repair. However, the CSA and/or CSB gene products may also play a role in clearing the stalled RNAP II molecule from the lesion site so that repair can occur and transcription resume (Hanawalt (2000) Nature 405:415-6; Mullenders (1998) Mutat. Res. 409:59-64). The CSB gene product is also critical for the repair of nucleotide base damage induced by reactive oxygen species (such as those generated by ionizing radiation or spontaneous metabolic processes) when such lesions are located on the transcribed strand of active genes (Leadon et al. (1993) Proc. Natl. Acad. Sci. (USA) 90:10499-503; Le Page et al. (2000) Cell 101:159-71). Furthermore, defects in TCR lead to sensitization to apoptosis induced by UV radiation, cisplatin, or ionizing radiation (Andera et al. (1997) Mol. Med 3:852-63; Chan et al. (1981) Mol. Gen. Genet. 181:562-3; Deschavanne et al. (1984) Mutat. Res. 131:61-70).

Cisplatin is a platinum compound which causes intra and interstrand covalent cross-linking of DNA leading to the formation of DNA adducts. It is regularly used to treat cervical, ovarian, head and neck and testicular cancer (Lokich et al. (1998) Ann. Oncol. 9:13-21). A major limitation to the prolonged use of cisplatin in all tumors is the development of resistance including up-regulation of DNA repair mechanisms that remove cisplatin-DNA adducts (Akiyama et al. (1999) Anticancer Drug Des. 14:143-51; Perez (1998) Eur. J. Can. 34:1535-42). De novo resistance is also a factor precluding the usefulness of cisplatin in lung and colorectal tumors (Raymond et al. (1998) Ann. Oncol 9:1053-71). Newer platinum drugs promise to change this. One important example, oxaliplatin, has a large spectrum of anti-tumor activity which is distinct from that of cisplatin, is less toxic to patients, and is highly effective against colorectal tumors that are typically resistant to cisplatin (de Gramont et al. (2000) J. Clin. Oncol. 18:2938-47; Misset et al. (2000) Crit. Rev. Oncol. Hematol. 35:75-93). Oxaliplatin is an analogue of cisplatin. (Cis [(1R, 2R) 1,2-cyclohexanediamine-N,N′ oxalato (2-)-O,O′] platinum). Even though oxaliplatin is effective against tumors resistant to cisplatin and thus must act differently from cisplatin in some way (Nehme et al. (1999) Br. J. Can. 79:1104-10), cisplatin and oxaliplatin both form mostly intrastrand DNA adducts which resemble UV-induced pyrimidine dimers (Woynarowski et al. (1998) Mol. Pharmacol. 54:770-7). In mammalian cells, both cisplatin and oxaliplatin-DNA adducts are removed by NER, the only mechanism known by which platinum-DNA intrastrand adducts are removed from DNA (Reardon et al. (1999) Can. Res. 59:3968-71).

NER deficiencies render cells more sensitive to cisplatin (Potapova et al. (1997) J. Biol. Chem. 272:14041-4; Pietras et al. (1994) Oncogene 9:1829-38; Arteaga et al. (1994) Can. Res. 54:3758-65; You et al. (1998) Oncogene 17:3177-86; Smith et al. (1996) Oncogene 13:2255-63; Koberle et al. (1999) Curr. Biol. 9:273-6) and elevated NER capacity is associated with resistance (States et al. (1996) Can. Lett. 108:233-7; Zeng-Rong et al. (1995) Can. Res. 55:4760-4; Chao (1996) Eur. J. Pharmacol. 305:213-22; Chao (1994) Eur. J. Pharmacol. 268:347-55; Eastman et al. (1988) Biochem. 27:4730-4). Intrastrand cisplatin adducts are known to induce the stalling of transcriptionally engaged RNAP II and to induce apoptosis, and are believed to play an important role in the cytotoxicity of these agents (Cullinane et al. (1999) Biochem. 38:6204-12). It was recently shown in a series of human ovarian carcinomas which became resistant to cisplatin that CSB mRNA levels were frequently increased (as were mRNA levels for the NER proteins XPA, XPB, and ERCC1), while mRNA levels of MDR1, another gene frequently associated with drug resistance, were not elevated (Dabholkar et al. (2000) Biochem. Pharmacol. 60:1611-1619).

Cisplatin and oxaliplatin also induce a small but significant number of interstrand cross-links (Jones et al. (1991) J. Biol. Chem. 266:7101-7; Trimmer et al. (1999) Essays Biochem. 34:191-211). Thus, NER is not sufficient to repair all platinum-induced DNA damage, and some studies suggest that the formation and repair of interstrand cross links may be the most informative factor for predicting cisplatin sensitivity (Zhen et al. (1992) Mol. Cell. Biol. 12:3689-98; Masumoto et al. (1999) Int. J. Canc. 80:731-7).

Given the ability of cancer cells to become resistant to chemotherapeutic and ionizing radiation approaches, the remains a need for new compounds and methods to overcome such resistance.

### BRIEF SUMMARY OF THE INVENTION

The invention provides methods, compositions, and formulations for potentiating or enhancing the toxicity of various cytotoxins and oxidizing agents, and of reducing the resistance and proliferation rate of cancer cells. It also provides various compositions and therapeutic formulations useful as anticancer agents.

The inventors have discovered that certain cytotoxins are more toxic to cells deficient in transcription coupled repair gene products or deficient in nucleotide repair gene products than to repair proficient cells. They have also determined that inhibiting NER or TCR potentiates the toxic effects of these cytotoxins. Additionally, the inventors have determined that cells can be sensitized to the toxic effects of oxidizing agents by contact with oligonucleotides directed to specific genes involved in NER or TCR.

These findings have been exploited to develop the present invention which, in one aspect, provides a method of potentiating or enhancing the toxic effect of a cytotoxin or an oxidizing agent on a cell. The method comprises contacting the cell with an oligonucleotide complementary to a gene involved in NER and/or TCR. The cell is then contacted with a toxic amount of a cytotoxin or an oxidizing agent. The toxic effect of the cytotoxin or oxidizing agent on the contacted cell is enhanced or potentiated after contact with the oligonucleotide.

As used herein, the term “potentiating” means increasing the length of time that a cytotoxin or oxidizing agent has an effect on a cell. The term “enhancing” is used herein to mean increasing, or making larger or stronger the effect of a cytotoxin or oxidizing agent on a cell. In some embodiments, the cell contacted is a carcinoma cell such as an ovarian, breast, or colon carcinoma cell in some embodiments.

The term “cytotoxin” as used herein encompasses compositions which poison a cell, resulting in its apoptosis or death. In particular embodiments, the cytotoxin used is selected from the group consisting of cisplatin, oxaliplatin, and analogs thereof. In one specific embodiment, the cytotoxin is cisplatin or oxaliplatin. A useful analog of cisplatin is carboplatin.

In certain particular embodiments, the oxidizing agent used is ionizing radiation, such as X-rays or gamma radiation.

In certain preferred embodiments, the oligonucleotide used to contact the cell is complementary to a portion of an NER or TCR gene selected from the group consisting of XPA, XPG, CSA, and CSB genes. In some preferred embodiments, the cell is contacted with an oligonucleotide directed to the CSB gene. In particular embodiments, the oligonucleotide is directed to the coding region of the CSB gene. In a particular embodiment, the oligonucleotide has a nucleotide sequence selected from the group consisting of SEQ ID NOS: 1 and 2. In preferred embodiments, the CSB-specific oligonucleotide used has phosphorothioate internucleotide linkages.

In other preferred embodiments, the cell is contacted with an oligonucleotide directed to the XPA gene. In particular embodiments, the oligonucleotide is directed to the coding region of the XPA gene. In a specific embodiment, the oligonucleotide has SEQ ID NO: 3. In another embodiment, the oligonucleotide is directed to the 3′-untranslated region of the XPA gene. In a specific embodiment, the oligonucleotide has SEQ ID NO:4. In preferred embodiments, the XPA-specific oligonucleotide used has phosphorothioate internucleotide linkages.

In yet other embodiments, the oligonucleotide used to contact the cell is directed to the coding or noncoding regions of the XPG or CSA genes.

In another aspect, the invention provides a method of sensitizing a resistant cell to a cytotoxin or an oxidizing agent. In this method, the cell is contacted with an oligonucleotide complementary to a gene involved in NER or TCR. The cell is then contacted with a cytotoxin or oxidizing agent in an amount that is toxic to a non-resistant cell. The contacted cell is less resistant to the cytotoxin or oxidizing agent after contact with the oligonucleotide.

The term “sensitizing” refers to the act of making a cell susceptible to or more affected by the effects of a compound or treatment. The term “resistant cell” encompasses cells that are not as affected by the toxic effects of a cytotoxin or oxidizing agent as is a “non-resistant cell.” Cells utilize a number of defense mechanisms to survive various toxins or treatments. Any agent that weakens such defense mechanisms will sensitize cells to the toxins or treatments. The sensitizing agent may not be toxic to the cell by itself.

In some embodiments, the cell contacted is a carcinoma cell such as an ovarian, breast, or colon carcinoma cell.

In particular embodiments, the cytotoxin used is selected from the group consisting of cisplatin and oxaliplatin. In one specific embodiment, the cytotoxin is cisplatin or oxaliplatin. In other particular embodiments, the oxidizing agent used is ionizing radiation such as X-rays or gamma radiation.

In preferred embodiments, the oligonucleotide used to contact the cell is complementary to a TCR or NER gene selected from the group consisting of XPA, XPG, CSA, and CSB genes. In some preferred embodiments, the cell is contacted with an oligonucleotide directed to the CSB gene. In particular embodiments, the oligonucleotide is directed to the coding region of the CSB gene. In a particular embodiment, the oligonucleotide has a nucleotide sequence selected from the group consisting of SEQ ID NOS: 1 and 2. In preferred embodiments, the CSB-specific oligonucleotide used has phosphorothioate internucleotide linkages.

In other preferred embodiments, the cell is contacted with an oligonucleotide directed to the XPA gene. In particular embodiments, the oligonucleotide is directed to the coding region of the XPA gene. In a specific embodiment, the oligonucleotide has SEQ ID NO:3. In another embodiment, the oligonucleotide is directed to the 3′-untranslated region of the XPA gene. In a specific embodiment, the oligonucleotide has SEQ ID NO:4. In preferred embodiments, the XPA-specific oligonucleotide used has phosphorothioate internucleotide linkages.

In yet other embodiments, the oligonucleotide used to contact the cell is directed to the coding or noncoding regions of the XPG or CSA genes.

In yet another aspect, the present invention provides a method of reducing the proliferation rate of a carcinoma cell, comprising contacting the cell with an oligonucleotide complementary to the CSB gene. As used herein, the term “reducing the proliferation rate” of a cell means slowing, stopping, or inhibiting the growth rate of cell.

In some embodiments, the cell is contacted with an oligonucleotide directed to the coding region of the CSB gene. In particular embodiments, the oligonucleotide has a nucleotide sequence selected from the group consisting of SEQ ID NOS: 1 and 2. In some embodiments, the oligonucleotide has phosphorothioate internucleotide linkages.

The invention also provides oligonucleotides complementary or directed to TCR or NER genes. In one aspect, the oligonucleotide is complementary to an XPA gene, the oligonucleotide having 20 to 50 nucleotides, and comprising SEQ ID NO:4 or SEQ ID NO:5. In a particular embodiment, the oligonucleotide has phosphorothioate internucleotide linkages.

In another aspect, the invention provides an oligonucleotide that is complementary to a CSB gene, the oligonucleotide having 20 to 50 nucleotides, and comprising SEQ ID NO: 1 or SEQ ID NO:2. In a particular embodiment, the oligonucleotide has phosphorothioate internucleotide linkages.

### DESCRIPTION OF THE DRAWINGS

The foregoing and other objects of the present invention, the various features thereof, as well as the invention itself, may be more fully understood from the following description, when read together with the accompanying drawing.

FIG. 1A is a graphic representation demonstrating that NER deficient fibroblasts show elevated sensitivity to oxaliplatin. Immortalized CS-A fibroblasts that were either restored to WT CSA status via stable transfection with the pDR2-CSA plasmid (pCSA) or stably transfected with the control pDR2 plasmid (cc) were subjected to oxaliplatin at the indicated doses for 3 days before relative proliferation was determined via MTS assay.

FIG. 1B is a graphic representation demonstrating that NER deficient fibroblasts show elevated sensitivity to cisplatin. Immortalized CS-A fibroblasts that were either restored to WT CSA status via stable transfection with the pDR2-CSA plasmid (pCSA) or stably transfected with the control pDR2 plasmid (cc) were subjected to cisplatin at the indicated doses for 3 days before relative proliferation was determined via MTS assay.

FIG. 1C is a graphic representation demonstrating that NER deficient fibroblasts show elevated sensitivity to oxaliplatin. Immortalized CS-B fibroblasts that were either restored to WT CSA status via stable transfection with the pDR2-CSB plasmid (pCSB) or stably transfected with the control pDR2 plasmid (cc) were subjected to oxaliplatin at the indicated doses for 3 days before relative proliferation was determined via MTS assay.

FIG. 1D is a graphic representation demonstrating that NER deficient fibroblasts show elevated sensitivity to cisplatin. Immortalized CS-B fibroblasts that were either restored to WT CSA status via stable transfection with the pDR2-CSB plasmid (pCSB) or stably transfected with the control pDR2 plasmid (cc) were subjected to cisplatin at the indicated doses for 3 days before relative proliferation was determined via MTS assay.

FIG. 2 is a graphic representation demonstrating that NER deficient fibroblasts show elevated sensitivity to oxaliplatin. Primary fibroblasts from XPA, XPG, or repair-competent individuals were exposed to oxaliplatin and assayed as described in FIGS. 1A-D.

FIG. 3 is a representation of a fluorescence image of an ethidium bromide stained gel demonstrating oligonucleotides reduce XPA and CSB mRNA levels. A2780/CP70 cells were transfected with the indicated oligonucleotides and then mRNA was isolated and subjected to rtPCR analysis. RtPCR products were resolved via agarose gel electrophoresis and visualized by ethidium bromide staining. For oligonucleotides targeting XPA mRNA, CSB mRNA was amplified as a control and for oligonucleotides targeting CSB mRNA, XPA mRNA was amplified as a control. Migration positions of 1000, 500, and 100 bp size markers are indicated at the right.

FIG. 4A is a graphic representation showing that oligonucleotides targeting CSB mRNA sensitize ovarian carcinoma cells to cisplatin. A2780/CP70 ovarian carcinoma cells were transfected with oligonucleotides HYB 969 (SEQ ID NO:1) or HYB 971 (SEQ ID NO:2) targeting CSB mRNA or control antisense oligonucleotide (HYB 1019) (SEQ ID NO:5) and then transferred to 96-well dishes for exposure to cisplatin at the indicated doses for three days followed by assessment of cell proliferation via MTS assay. (p=0.0007 for HYB 969 or 971 vs. oxaliplatin; p<0.0001 for HYB 969 or 971 vs. cisplatin).

FIG. 4B is a graphic representation showing that oligonucleotides targeting CSB mRNA sensitize ovarian carcinoma cells to cisplatin or oxaliplatin. A2780/CP70 ovarian carcinoma cells were transfected with oligonucleotides HYB 969 (SEQ ID NO: 1) or HYB 971 (SEQ ID NO:2) targeting CSB mRNA or control antisense oligonucleotide (HYB 1019) (SEQ ID NO:5) and then transferred to 96-well dishes for exposure to oxaliplatin at the indicated doses for three days followed by assessment of cell proliferation via MTS assay. (p=0.0007 for HYB 969 or 971 vs. oxaliplatin; p<0.0001 for HYB 969 or 971 vs. cisplatin).

FIG. 5A is a graphic representation demonstrating that oligonucleotides targeting XPA mRNA potentiates cisplatin toxicity. A2780/CP70 cells were transfected with oligonucleotide HYB 963 or oligonucleotide HYB 964 targeting XPA or oligonucleotide HYB 1040 (control) and 24 hours later were transferred to 96-well plates for assessment of sensitivity to cisplatin via MTS cell proliferation assay. (p<0.05 for HYB 963 vs. HYB 1040 for cisplatin treatment; p<0.01 for HYB 964 vs. HYB 1040 for cisplatin treatment).

FIG. 5B is a graphic representation demonstrating that oligonucleotides targeting XPA mRNA potentiates oxaliplatin toxicity. A2780/CP70 cells were transfected with oligonucleotide HYB 963 or oligonucleotide HYB 964 targeting XPA or oligonucleotide HYB 1040 (control) and 24 hours later were transferred to 96-well plates for assessment of sensitivity to cisplatin via MTS cell proliferation assay. (p<0.01 for HYB 963 or HYB 964 vs. HYB 1040 for oxaliplatin treatment.

FIG. 6 is a graphic representation demonstrating that oligonucleotides targeting XPA mRNA potentiates cisplatin toxicity. A2780/CP70 cells were transfected with HYB 964, HYB 1040 (control), or lipofectin alone (control) and 24 hours later transferred to soft agar. Cells were exposed to cisplatin or oxaliplatin at the indicated concentrations and colonies were counted ten days later. Asterisks indicate statistical comparison of HYB 964-transfected cells to HYB 1040-transfected cells (*,p<0.05,**, p<0.01).

FIG. 7A is a graphic representation showing that oligonucleotides targeting CSB mRNA sensitize ovarian carcinoma cells to oxidative damage. A2780/CP70 ovarian carcinoma cells were transfected with oligonucleotides HYB 971 (SEQ ID NO:2) targeting CSB mRNA or control antisense oligonucleotide (HYB 1019) (SEQ ID NO:5) and then transferred to 96-well dishes for exposure to hydrogen peroxide at the indicated concentrations, followed by three days of growth in normal medium and subsequent assessment of cell proliferation via MTS assay.

FIG. 7B is a graphic representation showing that oligonucleotides targeting CSB mRNA sensitize ovarian carcinoma cells to oxidative damage. A2780/CP70 ovarian carcinoma cells were transfected with oligonucleotides HYB 971 (SEQ ID NO:2) targeting CSB mRNA or control antisense oligonucleotide (HYB 1019) (SEQ ID NO:5) and then transferred to 96-well dishes for exposure to gamma radiation at the indicated doses followed by three days of growth in normal medium and subsequent assessment of cell proliferation via MTS assay.

FIG. 8 is a graphic representation showing that anti-CSB oligonucleotides retard cell proliferation in the absence of cytotoxic agents. A2780/CP70 ovarian carcinoma cells were transfected with indicated oligonucleotides and maintained in culture media for two more days to assess cell proliferation rate.

### DETAILED DESCRIPTION OF THE PREFERRED EMBODIMENTS

This invention relates to the fields of molecular biology and oncology. More particularly, this invention relates to the sensitization of cancerous cells to therapeutic agents.

The patent and scientific literature referred to herein establishes the knowledge that is available to those with skill in the art. The issued U.S. patents, allowed applications, published foreign applications, and references cited herein are hereby incorporated by reference. In the event of any conflict between any such document and the instant specification shall be resolved in favor of the latter.

The invention provides methods, compositions, and formulations for potentiating or enhancing the toxicity of various cytotoxins and oxidizing agents, and of reducing the resistance and proliferation rate of cancer cells. It also provides various compositions and therapeutic formulations useful as anticancer agents.

The inventors have discovered that certain cytotoxins are more toxic to cells deficient in transcription coupled repair gene products or deficient in nucleotide repair gene products than to repair proficient cells. They have also determined that inhibiting NER or TCR potentiates the toxic effects of these cytotoxins. Additionally, the inventors have determined that cells can be sensitized to the toxic effects of oxidizing agents by contact with oligonucleotides directed to specific genes involved in NER or TCR.

Standard reference works setting forth the general principles of the genetic and molecular biology technology described herein include Ott and Hoh, “Statistical Approaches to Genetic Mapping,” Am. J. Hum. Genet. 67:289-294 (2000); Zubay G., Genetics The Benjamin/Cummings Publishing Co., Inc., Menlo Park, Calif. (1987); Ausubel et al., Current Protocols in Molecular Biology, John Wiley & Sons, New York, N.Y. (1999); Sambrook et al., Molecular Cloning: A Laboratory Manual, 2d Ed., Cold Spring Harbor Laboratory Press, Plainview, N.Y. (1989); Kaufman et al. (Eds.), Handbook of Molecular and Cellular Methods in Biology and Medicine, CRC Press, Boca Raton, La. (1995); and McPherson, Ed., Directed Mutagenesis: A Practical Approach, IRL Press, Oxford (1991).

In the present invention, oligonucleotides are used to target NER or TCR gene products to reduce the level of target mRNA and potentiate or enhance the toxicity of various cytotoxins and oxidizing agents in cells treated with such cytotoxins and oxidizing agents. In addition, these oligonucleotides are useful for reducing the proliferation rate of the cancer cells even in the absence of treatment with cytotoxins or oxidizing agents.

The oligonucleotides according to the invention are complementary to a region of RNA, DNA or to a region of double-stranded DNA that encodes a portion of one or more genes involved in NER and/or TCR. The oligonucleotide can alternatively be directed to a non-coding region of such a gene.

For purposes of the invention, the term “oligonucleotide” includes polymers of two or more deoxyribonucleosides, ribonucleosides, or any combination thereof. Preferably, such oligonucleotides have from about 6 to about 50 nucleoside residues, and most preferably from about 12 to about 30 nucleoside residues. The nucleoside residues may be coupled to each other by any of the numerous known internucleoside linkages. Such internucleoside linkages include, without limitation, phosphorothioate, phosphorodithioate, alkylphosphonate, alkylphosphonothioate, phosphotriester, phosphoramidate, siloxane, carbonate, carboxymethylester, acetamidate, carbamate, thioether, bridged phosphoramidate, bridged methylene phosphonate, bridged phosphorothioate, and sulfone internucleotide linkages. These internucleoside linkages preferably are phosphotriester, phosphorothioate, or phosphoramidate linkages, or combinations thereof.

The oligonucleotides may also contain 2′-O-substituted ribonucleotides. For purposes of the invention, the term “2′-O-substituted” means substitution of the 2′ position of the pentose moiety with an —O-lower alkyl group containing 1-6 saturated or unsaturated carbon atoms, or with an —O-aryl or allyl group having 2-6 carbon atoms, wherein such alkyl, aryl, or allyl group may be unsubstituted or may be substituted, e.g., with halo, hydroxy, trifluoromethyl, cyano, nitro, acyl, acyloxy, alkoxy, carboxyl, carbalkoxyl, or amino groups; or such 2′ substitution may be with a hydroxy group (to produce a ribonucleoside), an amino or a halo group, but not with a 2′-H group. The term “alkyl,” as employed herein, refers to straight and branched chain aliphatic groups having from 1 to 12 carbon atoms, preferably 1-8 carbon atoms, and more preferably 1-6 carbon atoms, which may be optionally substituted with one, two or three substituents. Unless otherwise apparent from context, the term “alkyl” is meant to include saturated, unsaturated, and partially unsaturated aliphatic groups. When unsaturated groups are particularly intended, the terms “alkenyl” or “alkynyl” will be used. When only saturated groups are intended, the term “saturated alkyl” will be used. Preferred saturated alkyl groups include, without limitation, methyl, ethyl, propyl, isopropyl, butyl, isobutyl, sec-butyl, tert-butyl, pentyl, and hexyl.

The term oligonucleotide also encompasses such polymers having chemically modified bases or sugars and/or having additional substituents including, without limitation, lipophillic groups, intercalating agents, diamines, and adamantane. The term oligonucleotide also encompasses such polymers as PNA and LNA.

For purposes of the invention, the term “complementary” means having the ability to hybridize to a genomic region, a gene, or an RNA transcript thereof, under physiological conditions. Such hybridization is ordinarily the result of base-specific hydrogen bonding between complementary strands, preferably to form Watson-Crick or Hoogsteen base pairs, although other modes of hydrogen bonding, as well as base stacking can lead to hybridization. As a practical matter, such hybridization can be inferred from the observation of specific gene expression inhibition, which may be at the level of transcription or translation (or both). Useful oligonucleotides include chimeric oligonucleotides and hybrid oligonucleotides.

For purposes of the invention, a “chimeric oligonucleotide” refers to an oligonucleotide having more than one type of internucleoside linkage. One preferred embodiment of such a chimeric oligonucleotide is a chimeric oligonucleotide comprising internucleoside linkages, phosphorothioate, phosphorodithioate, internucleoside linkages and phosphodiester, preferably comprising from about 2 to about 12 nucleotides. Some useful oligonucleotides of the invention have an alkylphosphonate-linked region and an alkylphosphonothioate region (see e.g., Pederson et al. U.S. Pat. Nos. 5,635,377 and 5,366,878). Preferably, such chimeric oligonucleotides contain at least three consecutive internucleoside linkages that are phosphodiester and phosphorothioate linkages, or combinations thereof.

For purposes of the invention, a “hybrid oligonucleotide” refers to an oligonucleotide having more than one type of nucleoside. One preferred embodiment of such a hybrid oligonucleotide comprises a ribonucleotide or 2′-O-substituted ribonucleotide region, preferably comprising from about 2 to about 12 2′-O-substituted nucleotides, and a deoxyribonucleotide region. Preferably, such a hybrid oligonucleotide contains at least three consecutive deoxyribonucleosides and contains ribonucleosides, 2′-O-substituted ribonucleosides, or combinations thereof (see e.g., Metelev and Agrawal, U.S. Pat. Nos. 5,652,355 and 5,652,356).

The oligonucleotides of the invention, and used in the methods of the invention, are directed to any gene involved in TCR and/or NER. For purposes of the invention, a gene is “involved in” TCR and/or NER if the dininution of its expression abolishes or reduces the rate of TCR or NER. Over 20 genes are involved in NER. (see e.g. de Laat et al (1999) Genes & Dev. 13:768-85); ERCC1 (van Duin et al (1986) Cell 44:913-23; RPA 70 (Coverly et al (1991) Nature 349:538-41; RPA 32 (Coverly et al (1991) Nature 349:538-41; RPA 14 (Coverly et al (1991) Nature 349:538-41; hHR323B Mautani et al (1994) EMBO J 13:1831-43; TFIIH (p44 subunit) (Frit et al (1999) Biochimie 81:27-38; DNA polymerase delta; DNA polymerase epsilon; PCNA; RF-C (see Budd & Campbell, 1997, Mutat Res 384:157-67; Hindges & Hubscher 1997; Biol Chem 378:345-62; Jonsson & Hubscher 1997, BioEssays 19:967-75; Wood & Shivji, 1997 Carcinogenesis 18:605-10); DNA ligase I (Barnes et al (1992) Cell 69:495-503; Prigent et al (1994) Mol. Cell. Biol. 14:310-17); hMMS19 (Seroz et al (2000) Nucleic Acids Res. 28:4506-13; XAB2 is another TCR protein (Nakatsu et al (2000) JBC 275:34931-7).

Seven genes, XPA-XPG are known to be involved in TCR. These gene sequences are available on GenBank as follows:

XPA (XM_{—}009432 gi|11427749|ref|XM_{—}009432.1|[11427749]);

XPB (NM_{—}000122gi|4557562|ref|NM_{—}000122.1|[4557562]);

XPC (NM_{—}004628gi|4759331|ref|NM_{—}004628.1|[4759331]);

XPD (NM_{—}005236gi|4885216|ref|NM_{—}005236.1|[4885216]);

XPE (AJ002955gi|2632122|emb|AJ002955.1|HSAJ2955[2632122]);

XPF (XM_{—}007800gi|11430344|ref|XM_{—}007800.1|[11430344]) and

XPG (XM_{—}007128gi|12738017|ref|XM_{—}007128.2|[12738017]).

Useful oligonucleotides of the invention are directed to any of these genes. The nucleotide sequences of these genes are known in the art and are provided herein as SEQ ID NOS: 11, 12, 13, and 14, respectively. The oligonucleotides can be directed to the coding or non-coding regions of these genes.

Nonlimiting examples of oligonucleotides directed to the CSB gene are:

| | |
|---|---|
| HYB 969: | |
| 5′(2037)-d(GGTGACAGCAGCATTTGGAT)3′ | (SEQ ID NO:1) |
| and | |
| HYB 971: | |
| 5′-(3212)-d(GGAACATCATGGTCTGCTCC)-3′. | (SEQ ID NO:2) |

Nonlimiting examples of oligonucleotides directed to the XPA gene are:

| | |
|---|---|
| HYB 963: | |
| 5′(750)-d(GGTCCATACTCATGTTGATG)-3′ | (SEQ ID NO:3) |
| and | |
| HYB 964: | |
| 5′(1110)-d(CTGACCTACCACTTCTGCAC)-3′. | (SEQ ID NO:4) |

The exact nucleotide sequence and chemical structure of an antisense oligonucleotide utilized in the invention can be varied, so long as the oligonucleotide retains its ability to modulate expression of the target sequence. This is readily determined by testing whether the particular antisense oligonucleotide is active by quantitating the amount of mRNA encoding the gene, or quantitating the amount of NER or TCR, for example, to inhibit cell growth in an in vitro or in vivo cell growth assay, all of which are described in detail in this specification. The term “inhibit expression” and similar terms used herein are intended to encompass any one or more of these parameters.

Oligonucleotides according to the invention are useful for a variety of purposes, including potentiating or enhancing the toxic effects of oxidizing agents and cytotoxins on cells. They also can be used as “probes” of the physiological function of specific TCR- or NER-related proteins by being used to inhibit the activity of specific TCR- or NER-related proteins in an experimental cell culture or animal system and to evaluate the effect of inhibiting such specific TCR or NER activity. This is accomplished by administering to a cell or an animal an antisense oligonucleotide that inhibits one or more TCR or NER-related enzyme or other protein expression according to the invention and observing any phenotypic effects. In this use, the oligonucleotides used according to the invention are preferable to traditional “gene knockout” approaches because they are easier to use, and because they can be used to inhibit specific TCR- or NER-related protein activity.

Oligonucleotides according to the invention may conveniently be synthesized by any known method, e.g., on a suitable solid support using well-known chemical approaches, including H-phosphonate chemistry, phosphoramidite chemistry, or a combination of H-phosphonate chemistry and phosphoramidite chemistry (i.e., H-phosphonate chemistry for some cycles and phosphoramidite chemistry for other cycles). Suitable solid supports include any of the standard solid supports used for solid phase oligonucleotide synthesis, such as controlled-pore glass (CPG) (see, e.g., Pon (1993) Meth. Molec. Biol. 20:465-496).

The preparation of these modified oligonucleotides is well known in the art (reviewed in Agrawal (1992) Trends Biotechnol. 10:152-158; Agrawal et al. (1995) Curr. Opin. Biotechnol. 6:12-19). For example, nucleotides can be covalently linked using art-recognized techniques such as phosphoramidate, H-phosphonate chemistry, or methylphosphoramidate chemistry (see, e.g., Uhlmann et al. (1990) Chem. Rev. 90:543-584; Agrawal et al. (1987) Tetrahedron. Lett. 28:(31):3539-3542); Caruthers et al. (1987) Meth. Enzymol. 154:287-313; U.S. Pat. No. 5,149,798). Oligomeric phosphorothioate analogs can be prepared using methods well known in the field such as methoxyphosphoramidite (see, e.g., Agrawal et al. (1988) Proc. Natl. Acad. Sci. (USA) 85:7079-7083) or H-phosphonate (see, e.g., Froehler (1986) Tetrahedron Lett. 27:5575-5578) chemistry. The synthetic methods described in Bergot et al. (J. Chromatog. (1992) 559:35-42) can also be used.

The oligonucleotides of the invention are useful in various methods of the invention, including a method of potentiating or enhancing the toxic effects of a cytotoxin or oxidizing agent on a cancer cell. Cancer cells can be or become resistant to chemotherapeutic agents and oxidizing agents. The oligonucleotides of the invention sensitize such cells to these anticancer treatments. Cancer cells to be treated by the methods of the invention include any cells whose growth is uncontrolled including, but not limited to, ovarian, breast, and colon carcinoma cells. Cancer cells which are resistant to chemotherapeutic agents and/or radiation therapy respond particularly well to the methods of the invention.

According to the method of the invention, the cells are contacted with an oligonucleotide directed to NER or TCR-specific genes, and then are contacted with an amount of the cytotoxin or oxidizing agent that is toxic to unresistant cells.

Any cytotoxin known in the art to be useful for treatment of cancer is useful in the method of the invention. Particularly useful cytotoxins include platinum compounds that lead to the cross-linking of DNA. Useful platinum compounds include cisplatin, and analogs thereof, such as carboplatin, and oxaliplatin and analogs thereof. Both cisplatin and oxaliplatin induce intrastrand adducts subject to repair by NER, and defective NER increases the cytotoxicity of both agents. Cisplatin (CIS-diamminedichloroplatinum) can be commercially obtained, for example, from Bristol-Meters Squibb (Princeton, N.J.). Oxaliplatin (Cis [(1R, 2R) 1,2-cyclohexanediamine-N,N′ oxalato (2-)-O,O′] platinum) is available from NCI. Carboplatin is a cis platinum analogue, diamine[1,1′-cyclobutane -dicarboxylato(2-)-O,O′]-SP-4-2) (Paraplatin). The amount of cytotoxin to be administered to the cells in the methods of the invention can be determined by performing dose response experiments with cancerous cells that have not been treated with oligonucleotides directed to NER genes.

Ionizing radiation useful in the methods of the invention includes particulate and electromagnetic (photon) radiation such as X-rays and gamma rays, which causes breaks in DNA, resulting in cellular dysfunction and eventually, in cell death. Ionizing radiation can be provided by radionuclides or machines which generate radiation, as is well other sources known in the art. The amount of ionizing radiation to be administered to the cells in the methods of the invention can be determined by performing dose response experiments on cancerous cells that have not been treated with oligonucleotides directed to NER or TCR genes, using varying amounts of ionizing radiation.

The synthetic oligonucleotides of the invention directed to TCR or NER genes when in the form of a therapeutic formulation, are useful in treating diseases, disorders, and conditions associated with cancer. In such methods, a therapeutic amount of a synthetic oligonucleotide of the invention and effective in inhibiting the expression of a TCR or NER gene, in some instances with an oxidizing or cytotoxic agent, are administered to a cell. This cell may be part of a cell culture, a tissue culture, or may be part or the whole body of an animal such as a human or other mammal.

If the cells to be treated by the methods of the invention are in a subject, such as an animal, the oligonucleotides of the invention and the cytotoxins are administered as therapeutic compositions in pharmaceutically acceptable carriers. For example, cisplatin and its analogs, as well as other platinum compounds and cytotoxins can be administered to cancer patients as described by Slapak et al. in Harrison's Principles of Internal Medicine, 14^{th} Edition, McGraw-Hill, N.Y. (1998) Chapter 86.

Administration may be bolus, intermittent, or continuous, depending on the condition and response, as determined by those with skill in the art. In some preferred embodiments of the methods of the invention described above, the oligonucleotide is administered locally (e.g., intraocularly or interlesionally) and/or systemically. The term “local administration” refers to delivery to a defined area or region of the body, while the term “systemic administration” is meant to encompass delivery to the whole organism by oral ingestion, or by intramuscular, intravenous, subcutaneous, or intraperitoneal injection.

The synthetic oligonucleotides of the invention may be used as part of a pharmaceutical composition when combined with a physiologically and/or pharmaceutically acceptable carrier. The characteristics of the carrier will depend on the route of administration. Such a composition may contain, in addition to the synthetic oligonucleotide and carrier, diluents, fillers, salts, buffers, stabilizers, solubilizers, and other materials well known in the art. The pharmaceutical composition of the invention may also contain other active factors and/or agents which enhance inhibition of NER or TCR gene expression or which will reduce cancer cell proliferation. For example, combinations of synthetic oligonucleotides, each of which is directed to different regions of a TCR or NER gene mRNA, may be used in the pharmaceutical compositions of the invention. The pharmaceutical composition of the invention may further contain nucleotide analogs such as azidothymidine, dideoxycytidine, dideosyinosine, and the like. Such additional factors and/or agents may be included in the pharmaceutical composition to produce a synergistic effect with the synthetic oligonucleotide of the invention, or to minimize side-effects caused by the synthetic oligonucleotide of the invention. Conversely, the synthetic oligonucleotide of the invention may be included in formulations of a particular anti-TCR or NER gene or gene product factor and/or agent to minimize side effects of the anti-TCR or NER gene factor and/or agent.

The pharmaceutical composition of the invention may be in the form of a liposome in which the synthetic oligonucleotides of the invention are combined, in addition to other pharmaceutically acceptable carriers, with amphipathic agents such as lipids which exist in aggregated form as micelles, insoluble monolayers, liquid crystals, or lamellar layers which are in aqueous solution. Suitable lipids for liposomal formulation include, without limitation, monoglycerides, diglycerides, sulfatides, lysolecithin, phospholipids, saponin, bile acids, and the like. One particularly useful lipid carrier is lipofectin. Preparation of such liposomal formulations is within the level of skill in the art, as disclosed, for example, in U.S. Pat. No. 4,235,871; U.S. Pat. No. 4,501,728; U.S. Pat. No. 4,837,028; and U.S. Pat. No. 4,737,323. The pharmaceutical composition of the invention may further include compounds such as cyclodextrins and the like which enhance delivery of oligonucleotides into cells, as described by Zhao et al. (Antisense Res. Dev. (1995) 5:185-192), or slow release polymers.

As used herein, the term “therapeutically effective amount” means the total amount of each active component of the pharmaceutical composition or method that is sufficient to show a meaningful patient benefit, i.e., reducing the size of a tumor or inhibiting its growth or inhibiting the proliferation rate of cancer cells. When applied to an individual active ingredient, administered alone, the term refers to that ingredient alone. When applied to a combination, the term refers to combined amounts of the active ingredients that result in the therapeutic effect, whether administered in combination, serially or simultaneously.

In practicing the method of treatment or use of the present invention, a therapeutically effective amount of one, two, or more of the synthetic oligonucleotides of the invention is administered to a subject afflicted with a disease or disorder related to cancer. The synthetic oligonucleotide of the invention may be administered in accordance with the method of the invention either alone or in combination with oxidizing agents or cytotoxins, and/or other known therapies for cancer. When co-administered with one or more other therapies, the synthetic oligonucleotide of the invention may be administered either simultaneously with the other treatment(s), or sequentially. If administered sequentially, the attending physician will decide on the appropriate sequence of administering the synthetic oligonucleotide of the invention in combination with the other therapy.

Administration of the synthetic oligonucleotide of the invention used in the pharmaceutical composition or to practice the method of the present invention can be carried out in a variety of conventional ways, such as intraocular, oral ingestion, inhalation, or cutaneous, subcutaneous, intramuscular, or intravenous injection.

When a therapeutically effective amount of synthetic oligonucleotide of the invention is administered orally, the synthetic oligonucleotide will be in the form of a tablet, capsule, powder, solution or elixir. When administered in tablet form, the pharmaceutical composition of the invention may additionally contain a solid carrier such as a gelatin or an adjuvant. The tablet, capsule, and powder contain from about 5 to 95% synthetic oligonucleotide and preferably from about 25 to 90% synthetic oligonucleotide. When administered in liquid form, a liquid carrier such as water, petroleum, oils of animal or plant origin such as peanut oil, mineral oil, soybean oil, sesame oil, or synthetic oils may be added. The liquid form of the pharmaceutical composition may further contain physiological saline solution, dextrose or other saccharide solution, or glycols such as ethylene glycol, propylene glycol or polyethylene glycol. When administered in liquid form, the pharmaceutical composition contains from about 0.5 to 90% by weight of the synthetic oligonucleotide and preferably from about 1 to 50% synthetic oligonucleotide.

When a therapeutically effective amount of synthetic oligonucleotide of the invention is administered by intravenous, subcutaneous, intramuscular, intraocular, or intraperitoneal injection, the synthetic oligonucleotide will be in the form of a pyrogen-free, parenterally acceptable aqueous solution. The preparation of such parenterally acceptable solutions, having due regard to pH, isotonicity, stability, and the like, is within the skill in the art. A preferred pharmaceutical composition for intravenous, subcutaneous, intramuscular, intraperitoneal, or intraocular injection should contain, in addition to the synthetic oligonucleotide, an isotonic vehicle such as Sodium Chloride Injection, Ringer's Injection, Dextrose Injection, Dextrose and Sodium Chloride Injection, Lactated Ringer's Injection, or other vehicles as known in the art. The pharmaceutical composition of the present invention may also contain stabilizers, preservatives, buffers, antioxidants, or other additives known to those of skill in the art.

The amount of synthetic oligonucleotide in the pharmaceutical composition of the present invention will depend upon the nature and severity of the condition being treated, and on the nature of prior treatments which the patent has undergone. Ultimately, the attending physician will decide the amount of synthetic oligonucleotide with which to treat each individual patient. Initially, the attending physician will administer low doses of the synthetic oligonucleotide and observe the patient's response. Larger doses of synthetic oligonucleotide may be administered until the optimal therapeutic effect is obtained for the patient, and at that point the dosage is not increased further. It is contemplated that the various pharmaceutical compositions used to practice the method of the present invention should contain about 10 μg to about 20 mg of synthetic oligonucleotide per kg body or organ weight.

The duration of intravenous therapy using the pharmaceutical composition of the present invention will vary depending on the severity of the disease being treated and the condition and potential idiosyncratic response of each individual patient. Ultimately the attending physician will decide on the appropriate duration of intravenous therapy using the pharmaceutical composition of the present invention.

If oligonucleotides of the invention are administered locoregionally (e.g., intraperitoneal) as opposed to systemically, normal tissue uptake should be reduced. In addition, methods of encapsulating oligonucleotides in liposomes and targeting these liposomes to selected tissues by inserting proteins into the liposome surface can be utilized and are currently meeting with success (Pagnan et al. (2000) J. Natl. Can. Inst. 92:253-61; Yu et al. (1999) Pharm. Res. 16:1309-15).

The synthetic oligonucleotides of the invention directed to TCR or NER genes when in the form of a therapeutic formulation, are useful in treating diseases, disorders, and conditions associated with cancer. In such methods, a therapeutic amount of a synthetic oligonucleotide of the invention and effective in inhibiting the expression of a TCR or NER gene, in some instances with an oxidizing or cytotoxic agent, are administered to a cell. This cell may be part of a cell culture, a tissue culture, or may be part or the whole body of an animal such as a human or other mammal.

If the cells to be treated by the methods of the invention are in a subject, such as an animal, the oligonucleotides of the invention and the cytotoxins are administered as therapeutic compositions in pharmaceutically acceptable carriers. For example, cisplatin and its analogs, as well as other platinum compounds and cytotoxins can be administered to cancer patients as described by Slapak et al. in Harrison's Principles of Internal Medicine, 14^{th} Edition, McGraw-Hill, N.Y. (1998) Chapter 86.

Administration may be bolus, intermittent, or continuous, depending on the condition and response, as determined by those with skill in the art. In some preferred embodiments of the methods of the invention described above, the oligonucleotide is administered locally (e.g., intraocularly or interlesionally) and/or systemically. The term “local administration” refers to delivery to a defined area or region of the body, while the term “systemic administration” is meant to encompass delivery to the whole organism by oral ingestion, or by intramuscular, intravenous, subcutaneous, or intraperitoneal injection.

The synthetic oligonucleotides of the invention may be used as part of a pharmaceutical composition when combined with a physiologically and/or pharmaceutically acceptable carrier. The characteristics of the carrier will depend on the route of administration. Such a composition may contain, in addition to the synthetic oligonucleotide and carrier, diluents, fillers, salts, buffers, stabilizers, solubilizers, and other materials well known in the art. The pharmaceutical composition of the invention may also contain other active factors and/or agents which enhance inhibition of NER or TCR gene expression or which will reduce cancer cell proliferation. For example, combinations of synthetic oligonucleotides, each of which is directed to different regions of a TCR or NER gene mRNA, may be used in the pharmaceutical compositions of the invention. The pharmaceutical composition of the invention may further contain nucleotide analogs such as azidothymidine, dideoxycytidine, dideosyinosine, and the like. Such additional factors and/or agents may be included in the pharmaceutical composition to produce a synergistic effect with the synthetic oligonucleotide of the invention, or to minimize side-effects caused by the synthetic oligonucleotide of the invention. Conversely, the synthetic oligonucleotide of the invention may be included in formulations of a particular anti-TCR or NER gene or gene product factor and/or agent to minimize side effects of the anti-TCR or NER gene factor and/or agent.

The pharmaceutical composition of the invention may be in the form of a liposome in which the synthetic oligonucleotides of the invention are combined, in addition to other pharmaceutically acceptable carriers, with amphipathic agents such as lipids which exist in aggregated form as micelles, insoluble monolayers, liquid crystals, or lamellar layers which are in aqueous solution. Suitable lipids for liposomal formulation include, without limitation, monoglycerides, diglycerides, sulfatides, lysolecithin, phospholipids, saponin, bile acids, and the like. One particularly useful lipid carrier is lipofectin. Preparation of such liposomal formulations is within the level of skill in the art, as disclosed, for example, in U.S. Pat. No. 4,235,871; U.S. Pat. No. 4,501,728; U.S. Pat. No. 4,837,028; and U.S. Pat. No. 4,737,323. The pharmaceutical composition of the invention may further include compounds such as cyclodextrins and the like which enhance delivery of oligonucleotides into cells, as described by Zhao et al. (Antisense Res. Dev. (1995) 5:185-192), or slow release polymers.

As used herein, the term “therapeutically effective amount” means the total amount of each active component of the pharmaceutical composition or method that is sufficient to show a meaningful patient benefit, i.e., reducing the size of a tumor or inhibiting its growth or inhibiting the proliferation rate of cancer cells. When applied to an individual active ingredient, administered alone, the term refers to that ingredient alone. When applied to a combination, the term refers to combined amounts of the active ingredients that result in the therapeutic effect, whether administered in combination, serially or simultaneously.

In practicing the method of treatment or use of the present invention, a therapeutically effective amount of one, two, or more of the synthetic oligonucleotides of the invention is administered to a subject afflicted with a disease or disorder related to cancer. The synthetic oligonucleotide of the invention may be administered in accordance with the method of the invention either alone or in combination with oxidizing agents or cytotoxins, and/or other known therapies for cancer. When co-administered with one or more other therapies, the synthetic oligonucleotide of the invention may be administered either simultaneously with the other treatment(s), or sequentially. If administered sequentially, the attending physician will decide on the appropriate sequence of administering the synthetic oligonucleotide of the invention in combination with the other therapy.

Administration of the synthetic oligonucleotide of the invention used in the pharmaceutical composition or to practice the method of the present invention can be carried out in a variety of conventional ways, such as intraocular, oral ingestion, inhalation, or cutaneous, subcutaneous, intramuscular, or intravenous injection.

When a therapeutically effective amount of synthetic oligonucleotide of the invention is administered orally, the synthetic oligonucleotide will be in the form of a tablet, capsule, powder, solution or elixir. When administered in tablet form, the pharmaceutical composition of the invention may additionally contain a solid carrier such as a gelatin or an adjuvant. The tablet, capsule, and powder contain from about 5 to 95% synthetic oligonucleotide and preferably from about 25 to 90% synthetic oligonucleotide. When administered in liquid form, a liquid carrier such as water, petroleum, oils of animal or plant origin such as peanut oil, mineral oil, soybean oil, sesame oil, or synthetic oils may be added. The liquid form of the pharmaceutical composition may further contain physiological saline solution, dextrose or other saccharide solution, or glycols such as ethylene glycol, propylene glycol or polyethylene glycol. When administered in liquid form, the pharmaceutical composition contains from about 0.5 to 90% by weight of the synthetic oligonucleotide and preferably from about 1 to 50% synthetic oligonucleotide.

When a therapeutically effective amount of synthetic oligonucleotide of the invention is administered by intravenous, subcutaneous, intramuscular, intraocular, or intraperitoneal injection, the synthetic oligonucleotide will be in the form of a pyrogen-free, parenterally acceptable aqueous solution. The preparation of such parenterally acceptable solutions, having due regard to pH, isotonicity, stability, and the like, is within the skill in the art. A preferred pharmaceutical composition for intravenous, subcutaneous, intramuscular, intraperitoneal, or intraocular injection should contain, in addition to the synthetic oligonucleotide, an isotonic vehicle such as Sodium Chloride Injection, Ringer's Injection, Dextrose Injection, Dextrose and Sodium Chloride Injection, Lactated Ringer's Injection, or other vehicles as known in the art. The pharmaceutical composition of the present invention may also contain stabilizers, preservatives, buffers, antioxidants, or other additives known to those of skill in the art.

The amount of synthetic oligonucleotide in the pharmaceutical composition of the present invention will depend upon the nature and severity of the condition being treated, and on the nature of prior treatments which the patent has undergone. Ultimately, the attending physician will decide the amount of synthetic oligonucleotide with which to treat each individual patient. Initially, the attending physician will administer low doses of the synthetic oligonucleotide and observe the patient's response. Larger doses of synthetic oligonucleotide may be administered until the optimal therapeutic effect is obtained for the patient, and at that point the dosage is not increased further. It is contemplated that the various pharmaceutical compositions used to practice the method of the present invention should contain about 10 μg to about 20 mg of synthetic oligonucleotide per kg body or organ weight.

The duration of intravenous therapy using the pharmaceutical composition of the present invention will vary depending on the severity of the disease being treated and the condition and potential idiosyncratic response of each individual patient. Ultimately the attending physician will decide on the appropriate duration of intravenous therapy using the pharmaceutical composition of the present invention.

If oligonucleotides of the invention are administered locoregionally (e.g., intraperitoneal) as opposed to systemically, normal tissue uptake should be reduced. In addition, methods of encapsulating oligonucleotides in liposomes and targeting these liposomes to selected tissues by inserting proteins into the liposome surface can be utilized and are currently meeting with success (Pagnan et al. (2000) J. Natl. Can. Inst. 92:253-61; Yu et al. (1999) Pharm. Res. 16:1309-15).

In order that the invention described herein may be more fully understood, the following examples are set forth. It should be understood that these examples are for illustrative purposes only and are not to be construed as limiting the present invention in any manner.

### EXAMPLE 1

### Effect of Absence of CSA or CSB on Toxicity of Cisplatin or Oxaliplatin

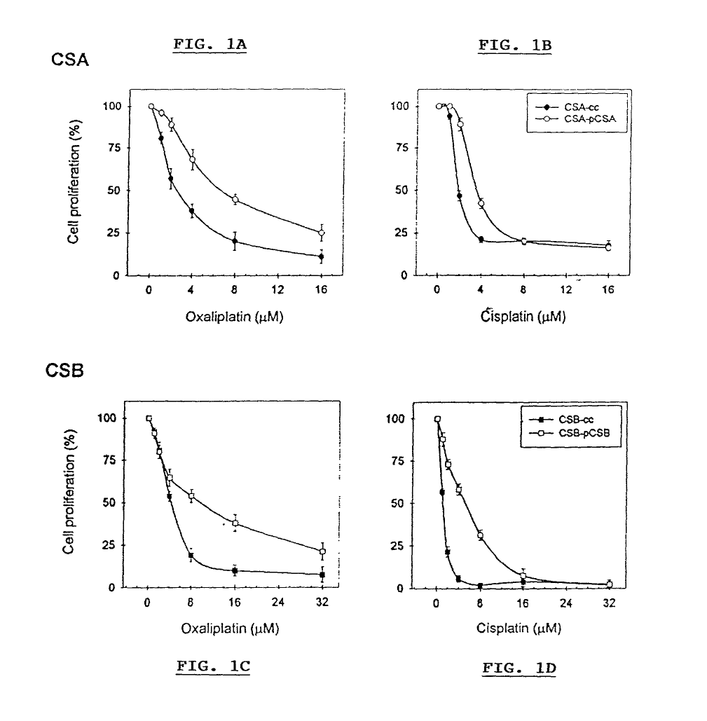

Since cisplatin adducts can induce RNAP II stalling (Cullinane et al. (1999) Biochem. 38:6204-12) and since the CSA and CSB gene products are known to help clear stalled RNAP II promoting transcriptional recovery after DNA damage, tests were done to determine whether fibroblasts which lacked functional CSA or CSB would be more sensitive to cisplatin or oxaliplatin. Immortalized CS-A and CS-B fibroblasts which have been restored to wild type (WT) status by the stable re-introduction of plasmid construct expressing the deficient CSA or CSB cDNA, respectively, have been characterized (Troelstra et al. (1992) Cell 71:939-953; Henning et al. (1995) Cell 82:555-564). Absence of either a functional CSA or CSB gene product rendered these virally transformed fibroblasts significantly more sensitive to either cisplatin or oxaliplatin (FIGS. 1A and 1B).

### EXAMPLE 2

### Effect of Absence of XPA or XPG on Toxicity of Cisplatin or Oxaliplatin

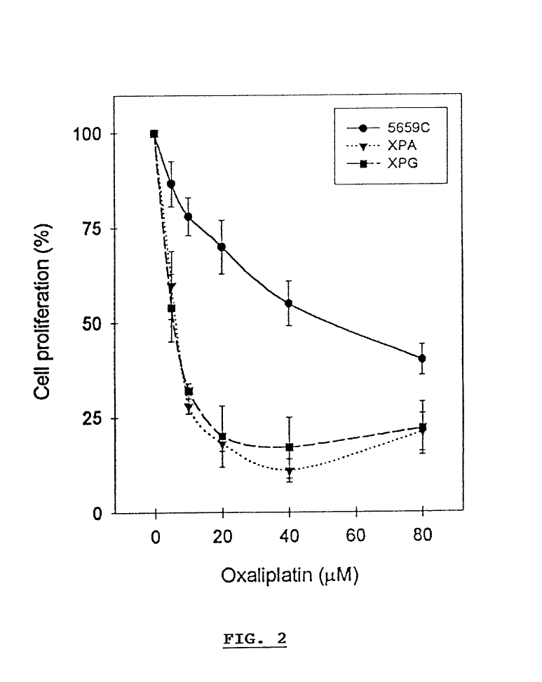

In addition, since NER-deficient XP cells are more sensitive to cisplatin, tests were done to determine whether XP-A and XP-G fibroblasts, two representative NER deficient cell lines were also more sensitive to oxaliplatin. XP-A and XP-G fibroblasts were significantly more sensitive to oxaliplatin (FIG. 2) as well as to cisplatin than were NER proficient 5659C fibroblasts.

### EXAMPLE 3

### Antisense Oligonucleotides as Potentiators of Cisplatin and Oxaliplatin

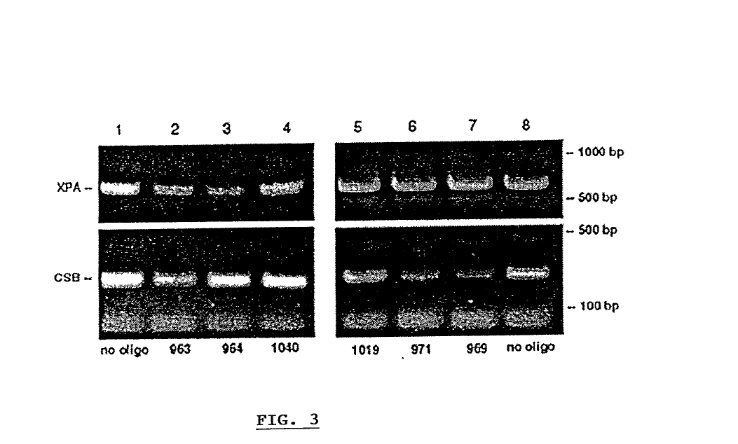

A panel of oligonucleotides (20 nucleotides in length) was synthesized that targeted the XPA and CSB mRNAs along their coding regions or their 5′ or 3′ noncoding regions. Oligonucleotides selected for further study were tested for their ability to reduce the levels of XPA or CSB mRNAs in A2780/CP70 ovarian carcinoma cells after they were introduced into these cells via transfection. Two oligonucleotides (HYB 963 and 964) which targeted the coding region of XPA mRNA and its 3′ untranslated region, respectively, were able to reduce XPA mRNA levels as determined by RT-PCR analysis (FIG. 3, lanes 2 and 3). A control antisense oligonucleotide (1040) did not reduce the level of XPA mRNA (FIG. 3, lane 4). Levels of a CSB mRNA were unchanged by any of the oligonucleotides targeting XPA sequences demonstrating that the levels of mRNA added to the assays were constant and that the oligonucleotides did not nonspecifically alter mRNA levels. Protein levels of XPA could also be reduced with anti-XPA oligonucleotides as determined by immunoblot analysis. Two oligonucleotides (HYB 969 and 971) which targeted the coding region of CSB mRNA were consistently able to reduce CSB mRNA levels in A2780/CP70 cells by about 50% (FIG. 3, lanes 6 and 7). A control antisense oligonucleotide (1019) did not reduce the level of CSB mRNA (FIG. 3, lane 5). Levels of XPA mRNA were unchanged by any of the oligonucleotides targeting CSB sequences demonstrating that the levels of mRNA added to the assays were constant and that the oligonucleotides did not nonspecifically alter mRNA levels.

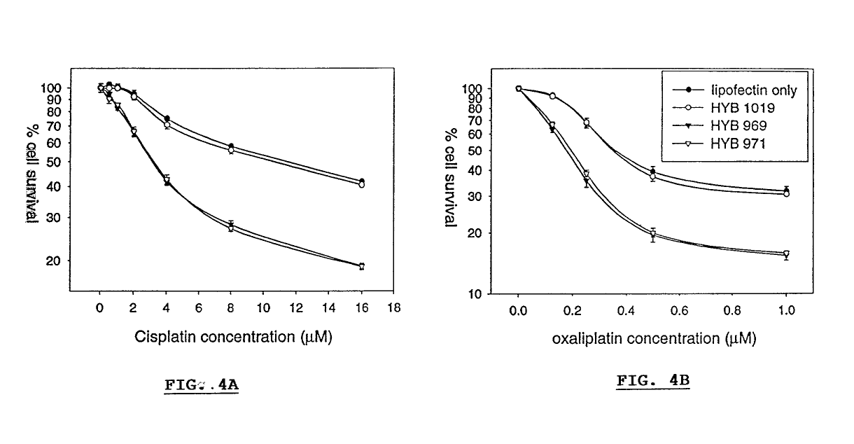

The oligonucleotides targeting CSB (969 and 971) were tested for their ability to sensitize A2780/CP70 cells to cisplatin or oxaliplatin. Cells were transfected with oligonucleotides and 24 hours later were replated on 96 well plates. After culturing in the presence of drug for another three days, cell viability was assessed by the MTS assay. Both oligonucleotides 969 and 971 substantially enhanced the cytotoxicity of both platinum agents (FIGS. 4A and 4B). In these experiments, 969 and 971 reduced the ID50 of cisplatin by 70% and the ID50 of oxaliplatin 50%. A non-hybridizing control antisense oligonucleotide (1019) did not alter the sensitivity of the cells to cisplatin or oxaliplatin. Oligonucleotides targeting CSB also potentiated cisplatin and oxaliplatin-induced cytotoxicity in SKBR3 breast cancer cells and HCT116 colon cancer cells.

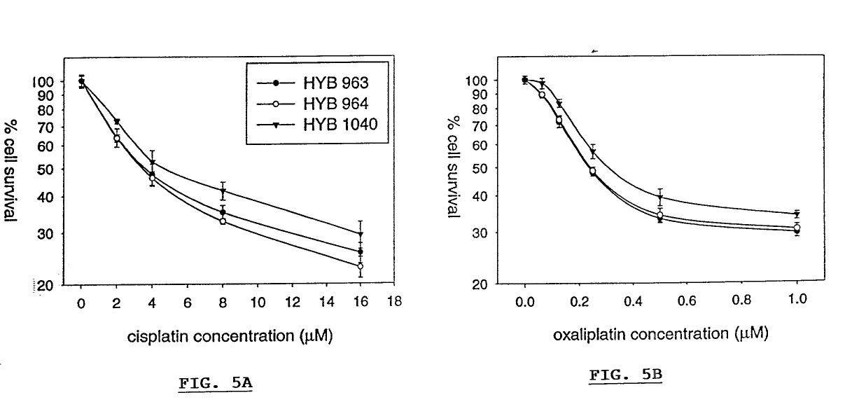

The oligonucleotides targeting XPA (HYB 963 and 964) were similarly tested for their ability to sensitize A2780/CP70 ovarian carcinoma cells to cisplatin or oxaliplatin. HYB 963 and 964 were able to increase the sensitivity of A2780/CP70 cells to cisplatin as well as oxaliplatin to a statistically significant degree albeit less robustly than did the oligonucleotides targeting CSB (FIGS. 5A and 5B). The oligonucleotides targeting XPA reduced the ID50 of oxaliplatin and cisplatin by about 25%.

### Example 4

### Antisense Oligonucleotides and Cisplatin or Oxaliplatin Inhibit Tumor Cell Proliferation

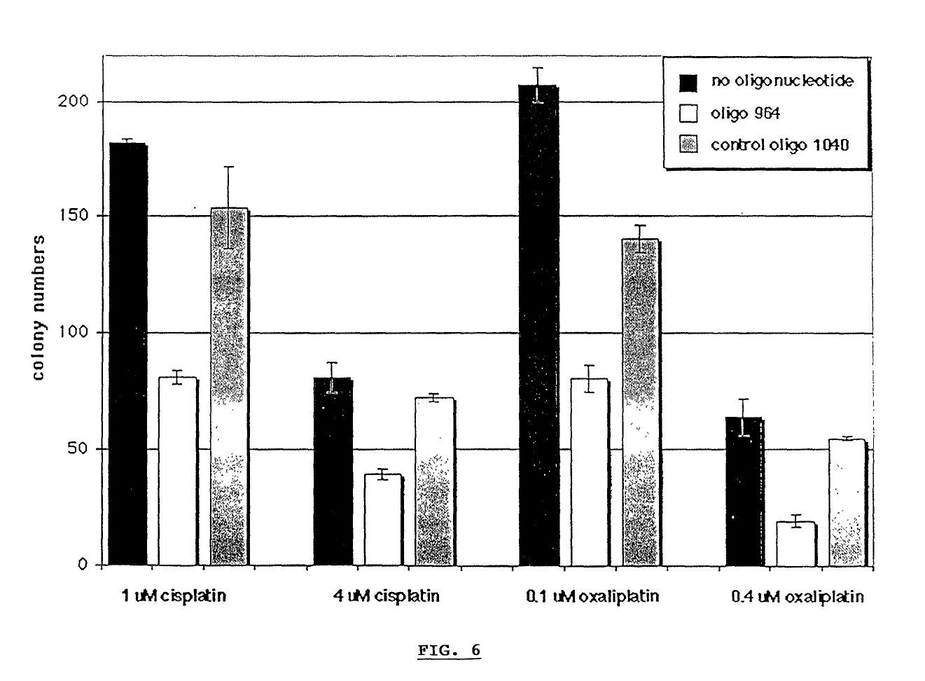

An alternative method for assessing the ability of oligonucleotides to inhibit tumor cell proliferation was also utilized. In this method, the transfected cells were transferred to soft agar containing various concentrations of oxaliplatin or cisplatin. Resulting colonies were counted 10 days later. Employing this assay, HYB 964 targeting XPA was shown to result in about 50% fewer colonies than either control HYB 1040 or lipofectin-only (sham) transfected cells (FIG. 6) in the presence of either cisplatin or oxaliplatin.

### Example 5

### CSB as a Target For Potentiating Cytotoxicity

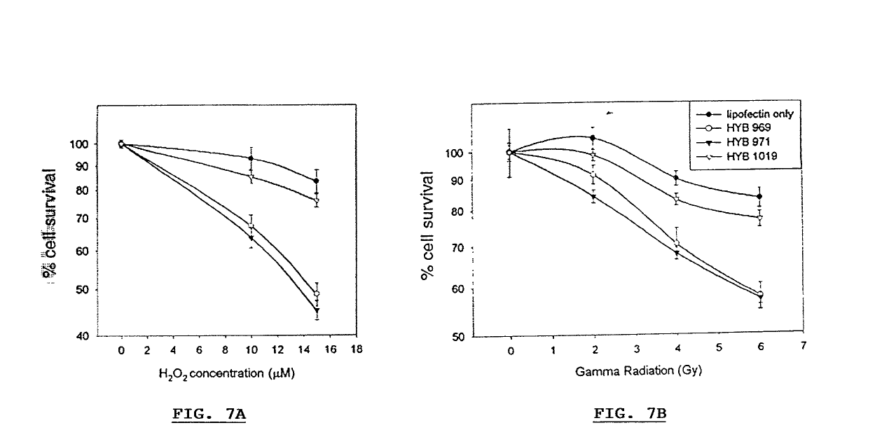

Tests were also performed to determine whether oligonucleotides targeting CSB could sensitize A2780/CP70 cells to oxidizing agents. Both HYB 969 and HYB 971 significantly increased the sensitivity of these cells to hydrogen peroxide (FIG. 7A) as well as gamma radiation (FIG. 7B).

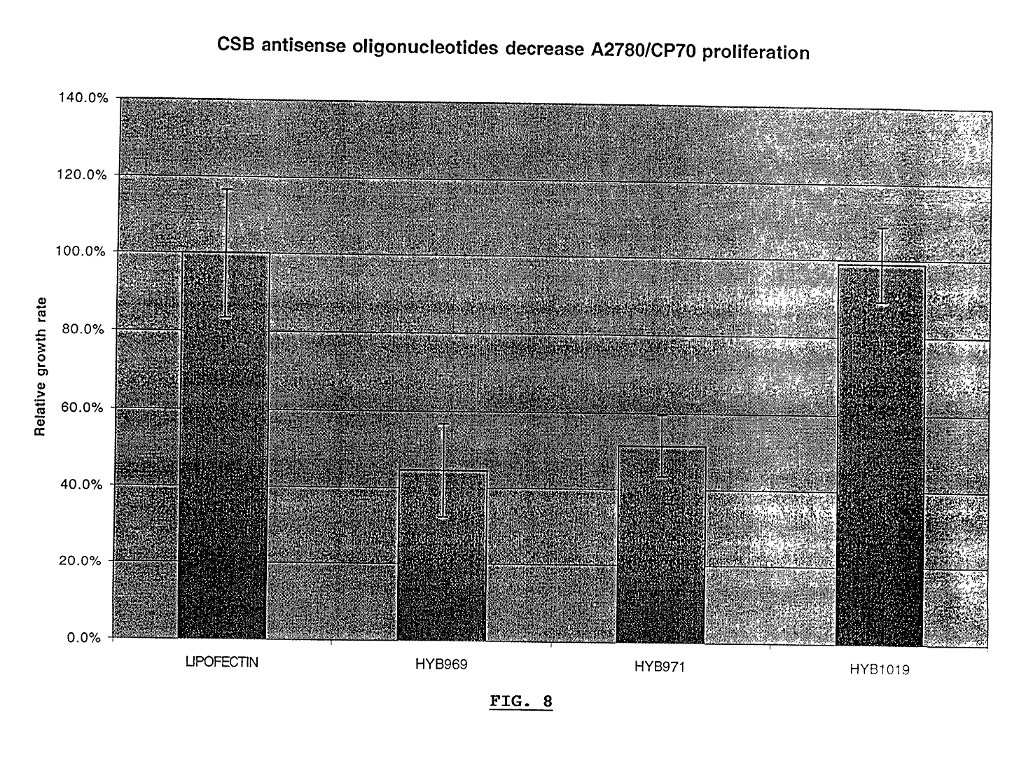

Tests were performed to measure the effect of oligonucleotides targeting CSB upon the proliferation of A2780/CP70 cells in the absence of any other anti-cancer agents. Both HYB 969 and HYB 971 reduced the proliferation of these cells by about 50% as compared to cells transfected with control antisense oligonucleotide (HYB 1019) sham transfected cells (FIG. 8).

It has been shown that disruption of the CSB gene in tumor predisposed Ink4a/ARF−/− mice reduces the number of spontaneous tumors and prolongs the latency period from 150 to 260 days despite the fact that these mice lack two tumor suppressor genes (Lu et al. (2001) Molec. Cell. Biol. (in press)). Mouse embryo fibroblasts (MEFs) derived from CSB−/−Ink4a/ARF−/− mice were significantly more susceptible to UV-induced apoptosis than Ink4a/ARF−/− MEFs. In addition, CSB-/-Ink4a/ARF-/-MEFs proliferated more slowly, demonstrated reduced mRNA synthesis rates, and demonstrated reduced immortalization potential via colony formation and ras transformation assays. These findings raised the possibility that disrupting the CSB gene could render cells more sensitive to DNA damaging anti-cancer agents. The results of the present study support this idea.

The ability of oligonucleotides targeting CSB to potentiate several DNA damaging anti-cancer agents could occur by blocking the cell's ability to clear stalled RNAP II from platinum adducts or from sites of oxidative DNA damage/repair (Le Page et al. (2000) Cell 101:59-71; Cullinane et al. (1999) Biochem. 38:6204-12). This is likely to promote apoptosis via p53 dependent as well as independent mechanisms (Lu et al. (2001) Molec. Cell. Biol. (in press); Yamaizumi et al. (1994) Oncogene 9:2775-2784; Ljungman et al. (1999) Oncogene 18:583-92; Ljungman et al. (1996) Oncogene 13:823-31). Furthermore, CSB deficiency may prevent recovery of mRNA synthesis which could in turn prevent progression to S phase (Mayne et al. (1982) Can. Res. 42:1473-8; Rocky et al. (2000) Proc. Natl. Acad. Sci. (USA) 97:10503-8; van Oosten et al. (2000) Proc. Natl. Acad. Sci. (USA) 97:11268-73).

An antiproliferative effect of CSB diminution by oligonucleotides occurs even in the absence of drug treatment (FIG. 8). This antiproliferative effect does not entirely account for the ability of oligonucleotides targeting CSB to potentiate cisplatin, oxaliplatin, hydrogen peroxide and ionizing radiation (FIGS. 4A, 4B, 7A, and 7B). When the cisplatin or oxaliplatin dose response curves for cells transfected with HYB 969 or 971 (the oligonucleotides targeting CSB) were normalized to values obtained from cells transfected with HYB 1019 (the control oligonucleotide), a robust potentiation by the oligonucleotides was still seen. Thus, although there was decreased proliferation in cells transfected with oligonucleotides targeting CSB even in the absence of cisplatin or oxaliplatin (FIG. 8), an additional effect upon cytotoxicity of these drugs definitely occurred.

### DETAILED MATERIALS AND METHODS

1. Cell Culture

The cisplatin-resistant ovarian carcinoma cell line A2780/CP70 was maintained in RPMI-1640 medium supplemented with 10% fetal bovine serum, 1× penicillin -streptomycin-neomycin (PSN) (Gibco, Rockville, Md.) 2 mM L-glutamine and 0.2 units/ml insulin (Novo Nordisk Pharmaceuticals, Princeton, N.J.) at 37_C under a humidified 5% CO₂ atmosphere. SV40-immortalized CS-B fibroblasts stably transfected with pCSB or control construct (generously provided by Dr. J. Hoeijmakers, Erasmus University, Rotterdam, Netherlands were maintained as previously described (Troelstra et al. (1992) Cell 71:939-953). SV40-immortalized CS-A cell lines (CS3BE.S3.G1+pDR2 and CS3BE.S3.G1+pDR2-CSA), were also maintained as described by Henning et al. (1995) Cell 82:555-564. DNA repair competent (GM 5659C), XP-A (GM2009), and XP-G (GM3021) fibroblasts were obtained from the National Institute of General Medical Sciences Human Genetic Mutant Cell Repository (Camden, N.J.) and maintained as described by Ratner et al. (1998) J. Biolog. Chem. 273:5184-5189. Gamma radiation was administered to cells in a 96 well plate with a Gamma Cell-40 Irradiator (Nordion International, Canada) while the 96 well plate was on ice.

2. Design and Synthesis of Oligonucleotides

Phosphorothioate oligonucleotides targeting XPA (Genbank Accession No. D14533) or CSB (Genbank Accession No. L04791) were designed based on the selection criteria described earlier (Agrawal et al. (2000) Mol. Med. Today 6:72-81). For each mRNA, 11 20-mer oligonucleotides targeting the coding region or noncoding regions of the molecule were designed. The oligonucleotides were synthesized on solid support with an automated DNA synthesizer using β(beta)-cyanoethylphos-phoramidite chemistry. Oxidation was carried out using Beaucage sulfurizing agent to obtain phosphorothioate backbone modified oligonucleotides. After the synthesis, oligonucleotides were released from the solid support, deprotected, purified by C18 reverse-phase HPLC, desalted, filtered, and lyophilized. The purity and sequence integrity of oligonucleotides was ascertained by capillary gel electrophoresis and MALDI-TOF mass spectral analysis, and the concentrations were determined by measuring absorbance at 260 nm.

3. Treatment of Cells with Oligonucleotides

Oligonucleotides were initially screened for their ability to potentiate cisplatin cytotoxicity in A2780/CP70 cells. The sequences of the two oligonucleotides against CSB selected for further study were:

| | |
|---|---|
| HYB 969: | |
| 5′(2037)-d(GGTGACAGCAGCATTTGGAT)-3′ | (SEQ ID NO:1) |
| HYB 971: | |
| 5′-(3212)-d(GGAACATCATGGTCTGCTCC)-3′. | (SEQ ID NO:2) |

The sequences of the three oligonucleotides targeting XPA selected for further study were:

| | |
|---|---|
| HYB 963: | |
| 5′(750)-d(GGTCCATACTCATGTTGATG)-3′ | (SEQ ID NO:3) |
| and | |
| HYB 964: | |
| 5′(1110)-d(CTGACCTACCACTTCTGCAC)-3′. | (SEQ ID NO:4) |

Nonhybridizing controls for CSB and XPA, respectively, were:

| | |
|---|---|
| HYB 1019: | |
| 5′(1612)-d(GCTACATAAGACCAGTGTGC)-3′ | (SEQ ID NO:5) |
| HYB 1040: | |
| 5′(590)-d(CCAAACCTGCACGATACATC)-3′. | (SEQ ID NO:6) |

which included 5-6 mismatched nucleotides.

Delivery of oligonucleotides into A2780/CP70 cells for RT-PCR and cell proliferation assays was achieved using Lipofectin (Life Technologies, Rockville, Md.) as per the manufacturer's procedure. The final concentration of oligonucleotides was 200 nM and final concentration of lipofectin was 10 μg/ml. After 4 hours incubation with the lipofectin-oligonucleotides mixture, cells were replaced with normal culture medium and treated as indicated for subsequent assays. A control FITC-labeled oligonucleotide (Sequitur, Natick, Mass.) was used to assess the delivery efficiency of oligonucleotides via lipofectin and demonstrated that about 50% of the cells successfully absorbed the FITC-labeled oligonucleotides into their nucleus.

4. RT-PCR Analysis

Total RNA was isolated from 2×10⁶ cells using a total RNA isolation kit (S.N.A.P., Invitrogen, Carlsbad, Calif.) as instructed and was quantitated spectrophotometrically via absorbance at 260 nm. RT-PCR analysis was performed using the Superscript One-Step RT-PCR System (Life Technologies, Rockville, Md.). Ten ng samples of total RNA were used for RT-PCR analyses because it was determined that quantities of RT-PCR products derived from XPA and CSB mRNA varied in a linear fashion when RT-PCR was performed on total RNA samples of 1-50 ng. For CSB, primers:

| | | |
|---|---|---|
| plus: | CCCTGCTGCACATCGACCGA | (SEQ 10 NO:7) |
| minus: | TGCCTTAGGGATGTCGTACA) | (SEQ ID NO:8) |

were selected to amplify a 235-bp segment.

For XPA, primers:

| | | |
|---|---|---|
| plus: | CAGGTCACTGAACTAAA | (SEQ 10 NO:9) |
| minus: | GGCTAATGTAAAAGCA) | (SEQ ID NO:10) |

were selected to amplify a 235-bp segment.

RT-PCR amplification was performed for 40 cycles to detect low mRNA levels while remaining in the linear range of PCR. Aliquots of amplified DNA were resolved via 1.5% agarose gel electrophoresis and visualized by ethidium bromide staining.

5. Cell Proliferation Assays

For A2780/CP70 cells transfected with oligonucleotides or mismatched controls, cells were harvested via trypsinization 16-24 hrs after transfection and transferred to 96 well plates at 5×10³ cells per well. To assay proliferation of fibroblasts with genetic NER defects or repair proficient fibroblasts (FIGS. 1A-D), cells were directly seeded onto 96 well plates at 5×10³ cells per well. More specifically, immortalized CS-A fibroblasts that were either restored to WT CSA status via stable transfection with the pDR2-CSA plasmid (pCSA) or stably transfected with the control pDR2 plasmid (cc) (Henning et al. (1995) Cell 82:555-564) were subjected to cisplatin or oxaliplatin. Twenty-four hours after transfer, cells in quadruplicate wells were treated with cisplatin (Sigma, St. Louis, Mo.) in 2 mM phosphate buffered saline (PBS) or oxaliplatin National Cancer Institute) in 4 mM PBS at serial dilutions in culture medium or with no drug and maintained for three more days. Cell survival was quantitated using the CellTiter 96 Non-radioactive Cell Proliferation Assay (Promega, Madison, Wis.). This is a colorimetric assay that quantitates living cells based on the principle that only metabolically active cells will convert 3-(4,5-dimethylthiazol-2-yl)-5-(3-carboxymethoxyphenyl)-2-(4-sulfophenyl)-2H-tetrazolium (MTS), a tetrazolium compound added to the culture medium, into a colored product (formazan) that can be detected via 490 nm absorbance using an ELx-800 microplate reader (Bio-Tek, Winooski, Vt.). A Trypan blue exclusion assay was also performed to verify that the values obtained via the cell titer assay correlated to numbers of viable cells. Readings from quadruplicate wells were averaged, normalized with respect to readings obtained from cells unexposed to drug, and are presented +/− standard deviation. Statistical significance was assessed via ANOVA (one-way followed by Dunnett's multiple comparison test) using the Prism software package (GraphPad, Inc. San Diego, Calif.). P values reported are for the multiple comparison test.

For growth in soft agar assay, cells transfected the previous day with oligonucleotides as described above were suspended (10⁴ cells/well) in 0.5 ml of 0.3% Difco Noble agar (Becton Dickinson & Co. Microbiology Systems, Sparks, Md.) supplemented with complete culture medium and layered over 0.5 ml of 0.8% agar-medium in chambers of 24 well plates. Drug was added (day 0) and colonies counted ten days later after staining with nitroblue tetrazolium (Sigma, St. Louis, Mo.) as previously described (Rockx, et al. (2000) Proc. Nat. Acad. Sci. (USA) 97:10503-8). For this assay, statistical comparison was via paired t-test.

A plate assay was also performed in the absence of added drug. Cells treated with oligonucleotides or mismatched controls were maintained in culture for two days. Cells were then trypsinized and cell number was determined using a hemacytometer. Numbers from three independent experiments were averaged and standard deviation was calculated. Statistical comparison was via paired t-test.

### EQUIVALENTS

The present invention is not to be limited in scope by the specific embodiments described herein. Indeed, various modifications of the invention, in addition to those described herein, will become apparent to those skilled in the art from the foregoing description and accompanying figures. Such modifications are intended to fall within the scope of the appended claims.

1. A method of potentiating or enhancing the toxic effect of a cytotoxin or an oxidizing agent on a cancer cell, comprising:
(A) contacting the cell with an oligonucleotide complementary to a gene selected from the group consisting of Xeroderma pigmentosum group A (XPA), Xeroderma pigmentosum group G (XPG), Cockayne syndrome group A (CSA), and Cockayne syndrome group B (CSB);
(B) contacting the cell with a toxic amount of an cytotoxin selected from the group consisting of cisplatin and oxaliplatin, or with a toxic amount of an oxidizing agent selected from the group consisting of ionizing radiation and hydrogen peroxide,
the toxic effect of the cytotoxin or oxidizing agent on the contacted cell being potentiated or enhanced after cellular contact with the oligonucleotide.

2. The method of claim 1, wherein the cytotoxin is cisplatin.

3. The method of claim 1, wherein the cytotoxin is oxaliplatin.

4. The method of claim 1, wherein the oxidizing agent is gamma radiation.

5. The method of claim 1, wherein the oxidizing agent is hydrogen peroxide.

6. The method of claim 1, wherein cell is contacted with an oligonucleotide directed to the CSB gene.

7. The method of claim 6, wherein the oligonucleotide is directed to the coding region of the CSB gene.

8. The method of claim 7, wherein the oligonucleotide has a nucleotide sequence selected from the group consisting of SEQ ID NOS: 1 and 2.

9. The method of claim 8, wherein the oligonucleotide has phosphorothioate internucleotide linkages.

10. The method of claim 1, wherein the oligonucleotide is directed to the XPA gene.

11. The method of claim 10, wherein the oligonucleotide is directed to the coding region of the XPA gene.

12. The method of claim 11, wherein the oligonucleotide has SEQ ID NO:3.

13. The method of claim 12, wherein the oligonucleotide has phosphorothioate internucleotide linkages.

14. The method of claim 10, wherein the oligonucleotide is directed to the 3′-untranslated region of the XPA gene.

15. The method of claim 14, wherein the oligonucleotide has SEQ ID NO:4.

16. The method of claim 15, wherein the oligonucleotide has phosphorothioate internucleotide linkages.

17. The method of claim 1, wherein the oligonucleotide is directed to XPG.

18. The method of claim 1, wherein the oligonucleotide is directed to CSA.

19. The method of claim 1, wherein the cell is a carcinoma cell.

20. The method of claim 19, wherein the carcinoma cell is selected from the group consisting of ovarian, breast, and colon carcinoma cells.

21. A method of sensitizing a resistant cell to a cytotoxin or an oxidizing agent, comprising:
(A) contacting the cell with an oligonucleotide complementary to a gene selected from the group consisting of Xeroderma pigmentosum group A (XPA), Xeroderma pigmentosum group G (XPG), Cockayne syndrome group A (CSA), and Cockayne syndrome group B (CSB);
(B) contacting the cell with a cytotoxin selected from the group consisting of cisplatin and oxaliplatin, or with an oxidizing agent selected from the group consisting of ionizing radiation and hydrogen peroxide,
the cell being contacted with an amount of cytotoxin or oxidizing agent that is cytotoxic to a non-resistant cell,
the contacted cell being less resistant to the cytotoxin or oxidizing agent after contact with the oligonucleotide.

22. The method of claim 21, wherein the cytotoxin is cisplatin.

23. The method of claim 21, wherein the cytotoxin is oxaliplatin.

24. The method of claim 21, wherein the oxidizing agent is gamma radiation.

25. The method of claim 21, wherein the oxidizing agent is hydrogen peroxide.

26. The method of claim 21, wherein cell is contacted with an oligonucleotide directed to the CSB gene.

27. The method of claim 26, wherein the oligonucleotide is directed to the coding region of the CSB gene.

28. The method of claim 27, wherein the oligonucleotide has a nucleotide sequence selected from the group consisting of SEQ ID NOS: 1 and 2.

29. The method of claim 28, wherein the oligonucleotide has phosphorothioate internucleotide linkages.

30. The method of claim 21, wherein the oligonucleotide is directed to the XPA gene.

31. The method of claim 30, wherein the oligonucleotide is directed to the coding region of the XPA gene.

32. The method of claim 31, wherein the oligonucleotide has SEQ ID NO:3.

33. The method of claim 32, wherein the oligonucleotide has phosphorothioate internucleotide linkages.

34. The method of claim 30, wherein the oligonucleotide is directed to the 3′-untranslated region of the XPA gene.

35. The method of claim 34, wherein the oligonucleotide has SEQ ID NO:4.

36. The method of claim 35, wherein the oligonucleotide has phosphorothioate internucleotide linkages.

37. The method of claim 21, wherein the oligonucleotide is directed to XPG.

38. The method of claim 21, wherein the oligonucleotide is directed to CSA.

39. The method of claim 21, wherein the cell is a carcinoma cell.

40. The method of claim 39, wherein the carcinoma cell is an ovarian, breast or colon carcinoma cell.

41. A method of reducing the proliferation rate of a carcinoma cell, comprising contacting the cell with an oligonucleotide complementary to the Cockayne syndrome group B (CSB) gene.

42. The method of claim 42, wherein the oligonucleotide is directed to the coding region of the CSB gene.

43. The method of claim 42, wherein the oligonucleotide has a nucleotide sequence selected from the group consisting of SEQ ID NOS: 1 and 2.

44. The method of claim 43, wherein the oligonucleotide has phosphorothioate internucleotide linkages.

45. An oligonucleotide complementary to a gene encoding Xeroderma pigmentosum group A (XPA), the oligonucleotide having 20 to 50 nucleotides, and comprising SEQ ID NO:4 or SEQ ID NO:5.

46. The oligonucleotide of claim 45 having phosphorothioate internucleotide linkages.

47. An oligonucleotide complementary to a gene encoding Cockayne syndrome group B (CSB), the oligonucleotide having 20 to 50 nucleotides, and comprising SEQ ID NO: 1 or SEQ ID NO:2.

48. The oligonucleotide of claim 47 having phosphorothioate internucleotide linkages.

49. A method of potentiating or enhancing the toxic effect of a cytotoxin or an oxidizing agent on a cancer cell, comprising contacting the cell with an oligonucleotide complementary to a gene involved in TCR and NER and contacting the cell with a toxic amount of a cytotoxin or an oxidizing agent.
